(19) United States
(12) Patent Application Publication
(10) Pub. No.: US 2008/0305558 A1
(43) Pub. Date: Dec. 11, 2008

(54) Cancer Screening Test

(76) Inventors: Ruth Louise Loveday, Hull (GB); John Greenman, Hull (GB)

Correspondence Address: RENNER OTTO BOISSELLE & SKLAR, LLP, 1621 EUCLID AVENUE, NINETEENTH FLOOR, CLEVELAND, OH, 44115, US

(21) Appl. No.: 12/158,233

(22) PCT Filed: Dec. 15, 2006
(86) PCT No.: PCT/GB2006/004721

§ 371 Date: Jun. 19, 2008

(30) Foreign Application Priority Data
Dec. 19, 2005 (GB) 0525754.8
Nov. 3, 2006 (GB) 0621904.2

(51) Int. Cl. G01N 33/543

(52) U.S. Cl. 436/518

(57) ABSTRACT

The invention provides a cancer screening test, to identify patients having an increased likelihood of cancer, comprising the step of determining the presence or absence of members of a test group of tumour associated antigens in the blood of a patient. The test group comprises a plurality of tumour-associated antigens. Antigens of particular interest include Vascular Endothelial Growth Factor-A (VEGF A); CEA 125 (Carcinoembryonic antigen 125); Prostate Specific Antigen (PSA); CA15-3 (Cancer antigen 15-3); CA125 (Cancer Antigen 125); CYFRA21-1 (Cytokeratin-19 fragments); Soluble ectodomain of c-erbB2; CA27.29 (Cancer Antigen 27.29); IGF-I (Insulin-like growth factor-1); IGF-2 (Insulin-like growth factor-2); and IGFBP-3 (Insulin-like growth factor binding protein 3). The invention also provides an antibody-based test kit to implement the screening method.

### FIELD OF THE INVENTION

The invention relates to a cancer screening test and a test kit for use in implementing the test. In particular, the test, and kit, have application in the identification of patients having an increased likelihood of cancer, especially breast, Lung, colorectal, prostate and ovarian cancer.

### BACKGROUND AND PRIOR ART KNOWN TO THE APPLICANT

In medicine, the usefulness of any diagnostic test or assay is assessed according to a number of criteria with reference to a “Gold Standard”, i.e. an accepted reference standard or diagnostic test for a particular illness. Typical criteria used are:
(1) Sensitivity: this is the probability of a test identifying a disease state among patients who have the disease, i.e. the proportion of people with the disease who wilt produce a positive test result.
(2) Specificity: this is the probability of the test giving a negative result for those patients who are free of the disease.
(3) Positive Predictive Value (PPV): this is defined as the percentage of people giving a positive test result who actually have the disease.
(4) Negative Predictive Value (NPV): this is the percentage of people with a negative test result who do not have the disease.

The qualities required for a useful clinical test depend on the context in which it is to be used. For example, if a test is to be used alongside other clinical markers, and in a patient population particularly at-risk from a particular condition, high sensitivity and specificity might not be required, as the test forms only part of an overall diagnostic schema. Tests might also be used to follow the response of a pre-diagnosed condition to therapeutic intervention. Clearly, in these instances, different qualities are required, as diagnosis is not the object.

However, when a test is destined to be used for mass screening of a general population, perhaps not showing other clinical signs, high specificity and sensitivity are paramount. In a study in 2003, Perkins et al [18] identify that screening tests require a high sensitivity in order to detect early-stage disease and that tests must also have sufficient specificity to protect patients with false-positive results from unwarranted diagnostic evaluations. To date, with the possible exception of prostate specific antigen (PSA) there is not a tumour marker identified that is sufficiently sensitive and specific enough to be used for screening purposes. Most tumour markers are instead implicated for use in monitoring patient response to drug treatment, as prognostic tools, and for the identification of metastases.

In general, approaches to the development of blood-based cancer screening tests have been considered using the observation that cancer patients have elevated levels of certain tumour-associated antigens in their blood stream. Although there is considerable research available on tumour associated antigens, there is not to date (with the exception of PSA) a tumour associated antigen that can be used for screening purposes on its own. A number of examples of attempts to use tumour-associated antigens are discussed below to exemplify the problem:

Carcinoembryonic antigen (CEA) was one of the first tumour markers to be identified and characterised [19, 20]. CEA is expressed in normal mucosal cells and is over-expressed in colorectal cancer, breast, lung, pancreatic and other cancers. Raised CEA in colorectal cancer has been correlated with advanced disease and it is generally believed that CEA is the best available non-invasive test for identifying recurrences during follow up of patients after surgery for colorectal cancer [21].

However, many studies have suggested that because of its lack of sensitivity in the early stages of colorectal cancer, CEA measurement is an unsuitable modality for population screening [22]. Although raised CEA in colorectal cancer has been correlated with advanced disease, it has also been demonstrated to be elevated in early stage disease. For example, Fernandes et al [23] demonstrated that the sensitivity at different stages was similar for stages I to III (23%-34%) and only higher in stage IV at 69%. Overall, the authors demonstrated that in the diagnosis of patients with colorectal adenocarcinoma, CEA showed a sensitivity of 56%, a specificity of 95%, a positive predictive value of 94%, a negative predictive value of 50%, and an accuracy of 76%.

CEA has also been reported to have prognostic value in patients with non-small cell lung cancer where it has demonstrated [24] a 52% specificity, and also in ovarian neoplasia [25]. In a study by Arslan et al, the serum CEA sensitivity and specificity in 172 patients with breast masses has been demonstrated to be 17% and 84% [26]. Again, therefore, as a tumour marker the sensitivity is too low to be of use in a screening assay.

Vascular endothelial growth factor has been proposed as a possible marker for detection of colorectal and other cancers. However, the variability and conflicting results of many studies have always militated against its use as a screening rule. For example, a study by Kumar et al [27] demonstrated that preoperative serum levels of VEGF can detect all but the very early stages of colorectal cancer and demonstrated that VEGF is a powerful predictor of outcome following curative surgery [28]. Other studies have supported this, and have demonstrated that elevated serum VEGF in colorectal cancer is associated with poor outcome [29, 30]. Previous work by the inventors has demonstrated that preoperative serum VEGF can detect breast cancer with a sensitivity of 62% and a specificity of 74%.

The significance of serum VEGF is supported by a number of other studies in several tumour types that have demonstrated serum VEGF levels to be higher than controls. These include colorectal [31], breast [32] and lung cancer [33]. However, and in contrast to these earlier studies, other work has not shown any significant difference between serum VEGF levels in colorectal cancer patients and control subjects [34]. Similarly, Granato et al [35] did not identify any significant difference in VEGF levels between cancer and control subjects in breast cancer.

In a study of VEGF serum concentrations of 122 colorectal cancer patients and 65 controls, Broll et al [36] demonstrated that VEGF was not a suitable diagnostic tumour marker due to its low sensitivity (36%).

Given the prime requirement of high sensitivity and high specificity for a mass-screening test, the information on tumour markers in the scientific literature, when taken as a whole, demonstrates that none is sufficiently sensitive or specific to be used for this purpose. So, although the promise of a screening methodology based on present tumour-associated antigens (and those still to be discovered) is alluring as a modality for mass population screening programmes, the skilled addressee, driven by the need for high sensitivity and specificity and operating with the caution required in the realm of public health policy, has always rejected their use for the problems and conflicting research findings discussed above.

Indeed, the only multiple cancer screening test approaching implementation is the DR-70 test (AMDL, USA). In this test, the developers rejected the use of tumour-associated antigens, and instead based the test on the detection of fibrin degradation products.

It is an object of the present invention to attempt a solution to some of these problems.

### SUMMARY OF THE INVENTION

Accordingly, the invention provides a cancer screening test, to identify patients having an increased likelihood of cancer, comprising the step of determining the presence or absence of members of a test group of tumour-associated antigens in the blood of a patient, said test group comprising a plurality of tumour-associated antigens; the presence, in the blood, of a plurality of antigens within said test group being indicative of patients having an increased such likelihood.

Although current research indicates that no one tumour-associated antigen shows sufficiently high sensitivity or specificity to be used as screening test, the inventors have found that testing for a combination of multiple positive results from a plurality of antigens leads to an overall test with sufficiently high sensitivity and specificity. Not only is the test able to provide a useful selection of patients requiring further investigation for specific cancers (see below), but also the screen is useful for a general indication of the presence of cancer.

Preferably, the plurality of tumour-associated antigens comprising the test group is associated with a plurality of cancer types. In this way, the screen is improved for the general indication of the presence of cancer.

Preferably also, the test group comprises a plurality of antigens selected from the group comprising: Vascular Endotheliat Growth Factor-A (VEGF A); CEA125 (Carcinoembryonic antigen 125); Prostate Specific Antigen (PSA); CA15-3 (Cancer antigen 15-3); CA125 (Cancer Antigen 125); CYFRA21-1 (Cytokeratin-19 fragments); Soluble ectodomain of c-erbB2; CA27.29 (Cancer Antigen 27.29); IGF-1 (Insulin-like growth factor-1); IGF-2 (Insulin-like growth factor-2); and IGFBP-3 (Insulin-like growth factor binding protein 3).

The inventors have found that these particular tumour-associated antigens render especially strong predictive power to the test.

In any aspect of the test, it is particularly preferred that the said test group comprises four or more antigens. The inventors have found that using at least four antigens raises the specificity and sensitivity of the test to a level where is has use in mass screening. For some applications where even more sensitivity and specificity is required, a test group comprising five, six or seven antigens is also particularly preferred.

The inventors have found that the use of such a combination of antigens is particularly effective as a screening test for particular cancer types, and so in any aspect of the invention, it is preferred that the test is to identify patients having an increased likelihood of one or more cancers selected from the group comprising: breast cancer; lung cancer; colorectal cancer; prostate cancer; and ovarian cancer.

Included within the scope of the invention is an assay kit for carrying out any of the screening tests described herein, the kit comprising antibodies capable of binding to each of the antigens in the test group. Preferably, the antibodies are bound to colloidal gold.

The invention thus provides a cancer screening test kit that will find use in general practice surgeries and health centres. It is envisaged that the test would be performed on patients over the age of 50; according to the UK's National Institute of Clinical Excellence (NICE) guidelines, this is the age range that would most benefit from cancer screening.

The principle behind the cancer screening kit is that cancer patients have elevated levels of certain tumour-associated antigens in their blood stream. Although research is available on tumour-associated antigens, the sensitivity and specificity of individual antigens is too low to provide a reliable screening test. No previous studies on the power of a combination of several tumour-associated antigens for screening purposes are known. The inventors have found that the use of a combination of tumour associated antigens significantly increases the sensitivity and specificity of such a test, the result and methodology having, for the first time, practical application for cancer screening.

The inventors have found that the detection of multiple markers for the same cancer leads to the production of fewer “false positive” results. Furthermore the detection of multiple markers, usually associated for a variety of different cancers, gives excellent predictive value for the general detection of cancer, i.e. not related to any particular tumour type.

The inventors have also found that the following tumour associated antigens are particularly useful for use in the method:
Vascular Endothelial Growth Factor-A (VEGF A) [1-4]
CEA125 (Carcinoembryonic antigen 125) [5,6]
Prostate Specific Antigen (PSA) [7]
CA15-3 (cancer antigen 15-3) [8,9]
CA125 (Cancer Antigen 125) [10]
CYFRA21-1 (Cytokeratin-19 fragments) [11]
Soluble ectodomain of c-erbB2 [12]
CA27.29 (Cancer Antigen 27.29) [13]
IGF-1 (Insulin-like growth factor-1) [14]
IGF-2 (Insulin-like growth factor-2) [15,16]
IGFBP-3 (Insulin-like growth factor binding protein 3) [17]

Whilst these particular antigens have been found to have particularly strong predictive value, it is envisaged that other tumour related antigens could also be incorporated into the test.

A particularly effective combination of tumour-related antigens has been developed, delivering high diagnostic power with a reduced number of markers. The antigens are given in Table 1. Also given in the table are Cut-Off Levels for each antigen. The presence of antigen at this concentration (or approximately so, say +/−10%), or higher, constituting a positive test for the presence of the antigen.

TABLE 1

| Antigen | Cut-Off Level |
|---|---|
| CEA | 5 ng/ml |
| CA15-3 | 30 U/ml |
| VEGF | 241 pg/ml |
| CA125 | 30 U/ml |
| c-erbB2 | 15 U/ml |
| IGF1 | 190 ng/ml |
| IGFII | 933 ng/ml |

### Implementation of the Test as a Kit

In one embodiment of a test assay kit, a format similar to that used for pregnancy screening tests is employed. Although used in a different field, this provides well-accepted, established and robust technology. The test device would comprise a sample window containing a wick impregnated with the antibodies of choice, together with a control antibody, bound to colloidal gold. On the addition a drop of the patient's blood to this region, the antibody-gold complex will bind to the tumour associated antigens of interest (if present) in the patient's blood, and then move by capillary transfer across the results window and bind to previously immobilised “capture antibodies”. The accumulation of colloidal gold at the capture sites will result in a coloured line appearing in the results window. A clinician will then be able to interpret the results to determine whether further clinical investigation is required.

### REFERENCES

1. Heer K, Kumar H, Read J R, Fox J N, Monson J R, Kerin M J. Serum vascular endothelial growth factor in breast cancer: its relation with cancer type and estrogen receptor status. Clinical Cancer Research (2001) 7: 3491-4

2. Ilhan N, Ilhan N, Deveci F. Functional significance of vascular endothelial growth factor and its receptor (receptor-1) in various lung cancer types. Clinical Biochemistry (2004) 37: 840-845

3. Kumar H, Heer K, Lee P W R, Duthie G S, MacDonald A W, Greenman J, Kerin M J, Monson J R T. Preoperative serum vascular endothelial growth factor can predict stage in colorectal cancer. Clinical Cancer Research (1998)4: 1279-1285

4. Cooper B, Ritchie J M, Broghammer C L W, Coffin J, Sorosky J I, Butler R E, Hendrix M J C, Sood A K. Preoperative serum vascular endothelial growth factor levels: significance in ovarian cancer. Clinical Cancer Research (2002) 8: 3193-3197

5. Fernandes L C, Kim S B, Matos D. Cytokeratins and carcinoembryonic antigen in diagnosis, staging and prognosis of colorectal adenocarcinoma. World J Gastroenterol. (2005)11: 645-8

6. Gebauer G, Muller-Ruchholtz. Tumor marker concentrations in normal and malignant tissues of colorectal cancer patients and their prognostic relevance. Anticancer Res. (1997) 17: 2939-42

7. Leach F S, Koh M S, Chan Y W, Bark S, Ray R, Morton R A, Rematey A T. Prostate specific Antigen as a Clinical Biomarker for Prostate Cancer: What's the take Home Message? Cancer Biol Ther. (2005) 4

8. Seregni E, Coli A, Mazzucca N. Circulating tumour markers in breast cancer. Eur J Nuclear Medicine and Molecular Imaging (2004) 31:15-21

9. Gion M, Mione R, Leon A E, Dittadi R. Comparison of the diagnostic accuracy of CA27.29 and CA15.3 in primary breast cancer. Clin Chem. (1999) 45: 630-7

10. Roupa Z, Raftopoulos V, Tzavelas G, Kotrotsious E, Sotiropoulou P, Karanikola E, Skifta E, Ardvanis A. Serum CA125 combined with transvaginal ultrasonography for ovarian cancer screening. In Vivo (2004) 18: 831-6

11. Nakata B, Takashima T, Ogawa Y, Ishikawa T, Hirakawa K. Serum CYFRA 21-1 (cytokeratin-79 fragments) is a useful tumour marker for detecting disease relapse and assessing treatment efficacy in breast cancer. British J Cancer (2004) 91: 873-878

12. Wu J T. c-erbB2 oncoprotein and its soluble ectodomain: a new potential tumor marker for prognosis early detection and monitoring patients undergoing Herceptin treatment. Clinical Chimica Acta (2002) 322: 11-19

13. Hou M F, Chen Y L, Tseng T F, Lin C M, Chen M S, Huang C J, Huang Y S, Hsieh J S, Huang T J, Jong S B, Huang Y F. Evaluation of serum CA27.29, CA75-5 and CEA in patients with breast cancer. Kaohsiung J Med Sci. (1999) 15: 520-8

14. Rinaldi S, Kaaka R, ZeLeniuch-Jacquotte A, Arslan A A, Shore R E, Koenig K L, Dossus L, Riboli E, Stattin P, Lukanova A, Toniolo P. Insulin-like growth factor-I, IGF binding protein-3, and breast cancer in young women: a comparison of risk estimates using different peptide assays. Cancer Epidemiol Biomarkers Prev. (2005) 14:48-52

15. Hunt K J, Toniolo P, Akhmedkhanov A, Lukanova A, Dechaud H, Rinaldi S, Zeleniuch-Jacquotte A, Shore R E, Riboli E, Kaaks R. Insulin-like Growth Factor II and Colorectal Cancer Risk in Women. Cancer Epidemiology, Biomarkers and Prevention (2002) 11: 901-905

16. Renehan A G, Jones J, Potten C S, Shalet SM, O'Dwyer ST. Elevated serum insulin-like growth factor (IGF)-II and IGF binding protein-2 in patients with colorectal cancer. British J Cancer (2000) 83: 1433-1450

17. Renehan A G, Zwahten M, Minder C, O'Dwyer S T, Shalet S M, Egger M. Insulin-like growth factor (IGF)-I, IGF binding protein-3, and cancer risk: systematic review and meta-regression analysis. Lancet (2004) 363: 1346-53

18. Perkins G L, Slater E D, Sanders G K, Prichard J G. Serum tumour markers. (2003) American Family Physician 68(6):1075-82

19. Guadagni F, Ferroni P, Carlini S, Mariotti S, Spila A, ALoe S, D'Alessandro R, Carone M D, Cicchetti A, Ricciotti A, Venturo I, Perri P, Filippo F D, Cognetti F, Botti C, Roselli M. A re-evaluation of CEA as a serum marker for breast cancer: a prospective longitudinal study. (2001) Clinical Cancer Research 7:2357-62

20. Sikorska H, Shuster J, Gold P. Clinical applications of CEA. (1988) Cancer Detect Prev. 12:321-55

21. Duffy M J. CEA as a marker for colorectal cancer: Is it clinically useful? (2001) Clin Chem. 47:624-630

22. Goldstein M J, Mitchell E P. Carcinoembryonic antigen in the staging and follow-up of patients with colorectal cancer. (2005) Cancer Invest. 23(4):338-51

23. Fernandes L C, Kim S B, Matos D. Cytokeratins and carcinoembryonic antigen in diagnosis, staging and prognosis of colorectal adenocarcinoma. (2005) World J Gastroenterol. 11: 645-48

24. Molina R, Filella X, Alicarte J, Zanon G, Pahisa J, Munoz M, Farrus B, Ballesta A M. Prospective evaluation of CEA and CA15.3 in patients with locoregional breast cancer. (2003) Anticancer Res. 23(2A):1035-41

25. Murta E F, da Silva C S, Gomes R A, Tavares-Murta B M, Melo A L. Ultrasonographic criteria and tumour marker assay are good procedures for the diagnosis of ovarian neoplasia in preselected outpatients. (2004) Eur J Gynaecol Oncol. 25(6):702-12

26. Arslan N, Serdar M, Deveci S, Ozturk B, Narin Y, Ilgan S, Ozturk E, Ozguven M A. Use of CA15-3, CEA and prolactin for the primary diagnosis of breast cancer and correlation with the prognostic factors at the time of initial diagnosis. (2000) Ann Nucl Med. 14(5):395-9

27. Kumar H, Heer K, Lee P W, Duthie G S, MacDonald A W, Greenman J, Kerin M J, Monson J R. Preoperative serum VEGF can predict stage in colorectal cancer. (1998) Clin Cancer Res. 4(5):1279-85

28. Chin K F, Greenman J, Gardiner E, Kumar H, Topping K, Monson J. Preoperative serum VEGF can select patients for adjuvant treatment after curative resection in colorectal cancer. (2000) Br J Cancer 83(11):1425-31

29. Werther K, Christensen I J, Brunner N, Nielsen H J. Soluble VEGF levels in patients with primary colorectal carcinoma. The Danish RANXO5 Colorectal Cancer Study Group. (2000) Eur J Surg Oncol. 26(7):657-62

30. Karayiannakis A J, Syrigos K N, Zbar A, Baibas N, Polychronidis A, Simopoulos C, Karatzas G. Clinical significance of preoperative serum VEGF levels in patients with colorectal cancer and the effect of tumour surgery. (2002) Surgery 131 (5):548-55

31.Broll R, Erdmann H, Windhovel U, Duchrow M, Strik M W. Determination of VEGF concentration in serum of patients with colorectal cancer. (1998) Langenbecks Arch Chir Suppl Kongressbd. 115(Suppl 1):315-18

32. Salven P, Perhoniemi V, Tykka H, Maenpaa H, Joensuu H. Serum VEGF levels in women with a benign breast tumour or breast cancer. (1999) Breast Cancer Res Treat 53(2):161-6

33. Kaya A, Ciledag A, Gulbay B E, Poyraz B M, Celik G, Sen E, Savas H, Savas I. The prognostic significance of vascular endothelial growth factor in sera of non-small cell lung cancer patient (2004) Respir Med. 98(7):632-36

34. Roumen R M, Slooter G D, Croiset van Uchelen F A, Huib L V. Preoperative serum VEGF is not a marker for subsequent recurrence during long-term follow-up of colorectal cancer patients. (2005) Dis Colon Rectum 48(5):1070-75

35. Granato A M, Nanni O, Falcini F, Folli S, Mosconi G, De Paola F, Medri L, Amadori D, Volpi A. Basic fibroblast growth factor and vascular endothelial growth factor serum levels in breast cancer patients and healthy women: useful as diagnostic tools? (2004) Breast Cancer Research 6(1):38-45

36. Broll R, Erdmann H, Duchrow M, Oevermann E, Schwander O, Markert U, Bruch H P, Windhovel U. VEGF-a valuable serum tumour marker in patients with colorectal cancer? (2001) Eur J Surg Oncot. 27(1):37-42

1. A cancer screening test, to identify patients having an increased likelihood of cancer, comprising the step of determining the presence or absence of members of a test group of tumour associated antigens in the blood of a patient, said test group comprising a plurality of tumour associated antigens;
the presence, in the blood, of a plurality of antigens within said test group being indicative of patients having an increased such likelihood.

2. A cancer screening test according to claim 1, wherein the plurality of tumour associated antigens comprising the test group is associated with a plurality of cancer types.

3. A cancer screening test according to either of claims 1 and 2 wherein said test group comprises a plurality of antigens selected from the group comprising:
Vascular Endothelial Growth Factor-A (VEGF A);
CEA125 (Carcinoembryonic antigen 125);
Prostate Specific Antigen (PSA);
CA15-3 (Cancer antigen 15-3);
CA125 (Cancer Antigen 125);
CYFRA21-1 (Cytokeratin-19 fragments);
Soluble ectodomain of c-erbB2;
CA27.29 (Cancer Antigen 27.29);
IGF-1 (Insulin-like growth factor-1);
IGF-2 (Insulin-like growth factor-2);
IGFBP-3 (Insulin-like growth factor binding protein 3).

4. A cancer screening test according to any preceding claim wherein said test group comprises four or more antigens.

5. A cancer screening test according to any preceding claim wherein the test is to identify patients having an increased likelihood of one or more cancers selected from the group comprising:
breast cancer;
lung cancer;
colorectal cancer;
prostate cancer; and
ovarian cancer.

6. An assay kit for carrying out the screening test of any preceding claim comprising antibodies capable of binding to each of the antigens in the test group.

7. An assay kit according to claim 6 wherein antibodies are bound to colloidal gold.
